(19) United States
(12) Patent Application Publication
(10) Pub. No.: US 2007/0277837 A1
(43) Pub. Date: Dec. 6, 2007

(54) Tissue protection and fluids collection mask for surgical applications and methods of use

(76) Inventor: Harkeerat Dhillon, Riverside, CA (US)

Correspondence Address: PATENT LAW & VENTURE GROUP, 2424 S.E. BRISTOL, SUITE 300, NEWPORT BEACH, CA, 92660, US

(21) Appl. No.: 11/447,478

(22) Filed: Jun. 5, 2006

(51) Int. Cl. A61F 11/00; A61B 19/00

(52) U.S. Cl. 128/863; 128/857

(57) ABSTRACT

A surgical mask is disk shaped and made of a flexible and elastic sheet material. It has a rim flange which strengthens the edge of the disk and prevents edge tears. An preferably off center linear slit is about 3 inches in length and provides terminal holes for preventing slit tear-out. In use, the mask is pressed down over a surgical site so as to be able to efficiently catch fluids erupting from the surgical site.

### BACKGROUND OF THE INVENTION

1. Field of the Present Disclosure

This disclosure relates generally to aprons or masks used for surgical applications and more particularly to a flexible plastic apron having a slit for placement over a surgical site for capturing viscous fluid runoff from the surgical site.

2. Description of Related Art Including Information Disclosed Under 37 CFR 1.97 and 1.98

Thompson et al. U.S. Pat. No. 5,002,069 discloses surgical drape which includes a pouch having a drawstring through a channel which runs along one edge of the pouch. When the drawstring is pulled through the channel, the edge of the pouch can be gathered to create a concave surface to control and contain fluids about a surgical site. Morris U.S. Pat. No. 4,974,604 discloses a surgical drape having a main sheet and a fluid collection system for collecting and draining away from the site of surgery substantial quantities of irrigating liquids. The fluid collection system has a bag means and a pair of fluid directing elements portions of which are secured to the drape in spaced-apart relationship near the primary operative area and remainder portions of which are secured to portions of the periphery of the bag. In one preferred embodiment, the fluid collection system is made by suitably folding and appropriately securing a single sheet of liquid impervious material. The main sheet is preferably a non-woven fabric. The liquid impervious sheet is preferably polyethylene. The fluid directing elements may have adhesive strips at their ends by which to secure the fluid collection system to a limb of a patient. The surgical drape may include a fenestrated region and having clipping tabs and tubing holders. The drape is particularly suited for use in arthroscopic surgery of the knee. Jackson U.S. Pat. No. 4,890,628 discloses a surgical drape with a fluid collection system for channeling and collecting fluids emanating from the surgical site during an operation. The surgical drape includes a main sheet with a fenestration located therein. A fluid collection bag is attached to the main sheet with its open end positioned adjacent the fenestration. The fluid collection bag has a front panel and a rear panel joined along common side edges to define a fluid receiving chamber. The front and rear panels further form a trough on either side of the bag extending upwardly from the open end along the common side edges. The fluid collection bag, with its integrally formed troughs, is attached to the main sheet such that the troughs and the open end of the fluid collection bag surround at least a portion of the fenestration with the troughs opening inwardly toward the fenestration so as to contain and channel fluids into the open end of the fluid collection bag. Idris U.S. Pat. No. 4,869,271 discloses a fenestrated surgical drape which has a fluid collection pouch pivotally attached about the fenestration so that the pouch may selectively attached to either side of the fenestration. As a result, the same surgical drape may be used for either right- or left-handed procedures. McAllester U.S. Pat. No. 4,598,458 discloses a surgical drape comprising an upper surface and a surgical site, and has a fluid collection bag attached to the upper surface. The bag has a first opening, a lower, fluid-collection chamber, and a second opening. The drape and bag are constructed such that when they are in their normal operational position, they are positioned such that the surgical site is above and in fluid communication with the first opening, the first opening is above and in fluid communication with the lower chamber, and the second opening is above and in fluid communication with the lower chamber. Neal U.S. Pat. No. 4,570,628 discloses a surgical drape composed of a main sheet of sheet material adapted for use by a patient in the lithotomic position. The surgical drape is fabricated from a main sheet of sheet material which is constructed of sufficient softness and is sufficiently inexpensive to be disposable. The main sheet is formed of a torso section and a bottom section which are interconnected by a pair of side sections. Each side is formed into an enclosed area so as to function as a legging for the patient. The main sheet includes a fenestration centrally located therein with a fenestration strengthening material located entirely about said fenestration. The fenestration strengthening material is to resist tearing and cutting. A layer of non-liquid absorbing protective material is attached to the fenestration strengthening material. A collection container in the form of a flexible walled bag is located adjacent the bottom section. The collection material is adapted to collect blood and solids which are discharged during the medical procedure. Volumetric measuring indicia are carried in the exterior wall of the bag to facilitate the ascertaining of the approximate loss of blood by the patient. In another construction, a second container is provided for collection of fecal matter and a multiplicity of slots, pockets and holders are employed for yieldably retaining a variety of medical implements and supplies.

Our prior art search described above teaches the use of surgical linens which are used as aprons and masks at the surgical site for capturing and absorbing fluids such as those natural fluids from the surgical site as well as fluids used for irrigation and antiseptic purposes. However, the prior art fails to teach a simple fluids collection mask having a slit within it that may be pressed down onto a surgical site so that the edges of the mask are in intimate contact with the surrounding areas and so that overflowing viscous fluids such as adhesives may be captured for disposal. The present disclosure distinguishes over the prior art providing heretofore unknown advantages as described in the following summary.

### BRIEF SUMMARY OF THE INVENTION

This disclosure teaches certain benefits in construction and use which give rise to the objectives described below.

The present apparatus is a disk shaped surgical mask made of a flexible and elastic sheet material. It has a rim flange which strengthens the edge of the disk and prevents edge tears. A preferably off center linear slit is about 3 inches in length and provides terminal holes for preventing slit tear-out. In use, the mask is pressed down over a surgical site so as to be able to efficiently catch fluids erupting from the surgical site. An important use of the present apparatus is to capture fluid adhesives that when injected into a surgical site, typically overflow causing a problem with the surgical surrounds (surrounding areas to the surgical site). The present apparatus provides the flexibility, sealing capacity and appropriate surface for capturing such adhesive fluids.

A primary objective inherent in the above described apparatus and method of use is to provide advantages not taught by the prior art.

Another objective is to provide a tissue protection and collection mask that is able to fully surround a surgical site to efficiently capture a fluid overflow.

A further objective is to provide such a mask that is chemically resistant and highly flexible.

A still further objective is to provide such a mask that is of an extremely thin sheet material having a consistency that is easily pressed against a surgical surround to form a tight seal therebetween.

Other features and advantages of the described apparatus and method of use will become apparent from the following more detailed description, taken in conjunction with the accompanying drawings, which illustrate, by way of example, the principles of the presently described apparatus and method of its use.

### BRIEF DESCRIPTION OF THE SEVERAL VIEWS OF THE DRAWING(S)

Illustrated in the accompanying drawing(s) is at least one of the best mode embodiments of the present invention In such drawing(s):

FIG. 1 is a perspective view of the present apparatus;

FIG. 2 is a sectional view thereof taken along line 2-2 in FIG. 1; and

FIG. 3 is a plan view thereof showing the apparatus in use.

### DETAILED DESCRIPTION OF THE INVENTION

The above described drawing figures illustrate the described apparatus and its method of use in at least one of its preferred, best mode embodiment, which is further defined in detail in the following description. Those having ordinary skill in the art may be able to make alterations and modifications to what is described herein without departing from its spirit and scope. Therefore, it must be understood that what is illustrated is set forth only for the purposes of example and that it should not be taken as a limitation in the scope of the present apparatus and method of use.

The presently described apparatus is a tissue protection and fluids collection mask apparatus for surrounding a surgical site so as to be able to collect fluids erupting from the site. A disk 10 of thin flexible and elastic sheet material provides a rim flange 20 which strengthens the edge of the disk so as to prevent tears. A linear slit 30 in the disk 10 includes a means for preventing slit tear-out 32 at both of its ends. This preventing means 32 preferably includes two round holes located at opposing ends of the linear slit 30. Preferably these holes are no larger than ¼ inch in diameter which has been found to be critical in preventing liquids from seeping below the mask. Preferably, the elastic sheet material is a natural latex or silicone rubber having a maximum thickness of 3/32″ which has been found to be critical in achieving the intimate contact and sealing properties between the apparatus and the surgical surround. Also, natural latex or silicone rubber has been found to provide high friction between itself and a skin surface so that the mask does not tend to slide away from the surgical site; a critically important feature. Preferably, the disk is between 12 and 20 inches in diameter which has been found to be critical to the easy handling and quick moving and placement of the apparatus during surgical procedures. Disks larger or smaller than this tend to be much less useful in capturing liquids and in being conveniently placed.

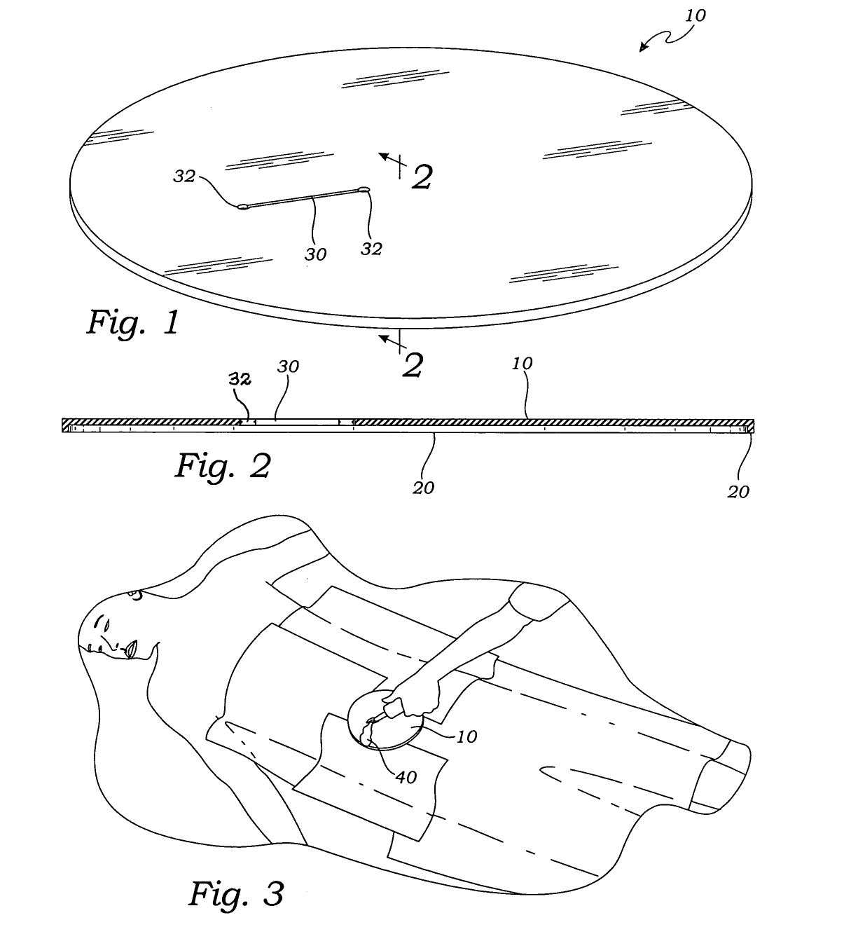

The slit 30 is preferably between 2.5 and 3.5 inches in length, and less than 3/32 inches in width, which has been found to be critical in providing fluid capture from a surgical site wherein the surgeon's hand is large enough to press the edges of the slit into a sealed contact with the surgical surround. A slit of lesser length does not provide enough of an opening for a typical surgical site and one of greater length is not able to be managed by the surgeon's hand alone so that leaks occur. Preferably, the slit is positioned on a diameter of the disk, i.e., medially, and away from the geometric center of the disk, i.e., slightly toward one edge, as shown in FIG. 1 because this slit orientation has been found to be of advantage in most surgical situations. This allows the bulk of the mask to lie below or “down-hill” from the surgical site so a to capture the fluid effectively.

Clearly, it is not required that the mask be circular, although that shape is preferable. Other shapes that are useful in the present apparatus are square, oval, rectangular, hexagonal and polygonal. Therefore, the word “disk” should be taken herein to mean any shape that is desirable for the intended purpose.

The rim flange 20 is preferably not greater than 9/32 inches in thickness which is critical to the ability of the mask to lay flat on the surgical site and yet prevent tearing of the very thin and somewhat fragile nature of the fabric of the mask.

In use, the apparatus is used for protecting the tissues from surgical debris and collecting fluids from a surgical site. The disk 10 is prepared of flexible and elastic sheet material and with the physical features described above. The disk 10 is draped over the surgical site with the slit opened around the surgical site and the slit as well as the bulk of the disk surface positioned in a down-hill direction from the surgical site. The edges of the slit are sealed against the surgical surround manually, while applying a fluid to the surgical site. Overflow of the fluid 40 from the surgical site is captured onto the disk 10 for disposal. When overflow ceases, the mask may be removed and is then discarded properly within the surgical theatre.

The enablements described in detail above are considered novel over the prior art of record and are considered critical to the operation of at least one aspect of the apparatus and its method of use and to the achievement of the above described objectives. The words used in this specification to describe the instant embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or drawing elements described herein are meant to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements described and its various embodiments or that a single element may be substituted for two or more elements in a claim.

Changes from the claimed subject matter as viewed by a person with ordinary skill in the art, now known or later devised, are expressly contemplated as being equivalents within the scope intended and its various embodiments. Therefore, obvious substitutions now or later known to one with ordinary skill in the art are defined to be within the scope of the defined elements. This disclosure is thus meant to be understood to include what is specifically illustrated and described above, what is conceptually equivalent, what can be obviously substituted, and also what incorporates the essential ideas.

The scope of this description is to be interpreted only in conjunction with the appended claims and it is made clear, here, that each named inventor believes that the claimed subject matter is what is intended to be patented.

What is claimed is:

1. A tissue protection and fluid collection mask apparatus comprises: a disk of flexible and elastic sheet material having high surface friction against a skin surface and providing a peripheral rim flange lending strength to a peripheral edge of the disk; and a linear slit positioned in the disk away from the peripheral edge, the slit incorporating, at the opposing ends thereof, a means for preventing slit tear-out.

2. The apparatus of claim 1 wherein the tear-out preventing means comprises two round holes, the holes located at opposing ends of the linear slit.

3. The apparatus of claim 2 wherein the holes are no larger than ¼ inch in diameter.

4. The apparatus of claim 1 wherein the elastic sheet material is one of a natural latex and a silicone rubber having a maximum thickness of 3/32″.

5. The apparatus of claim 1 wherein the disk is between 12 and 20 inches in diameter.

6. The apparatus of claim 1 wherein the slit is between 2.5 and 3.5 inches in length, and between 1/32 and 3/32 in width; the slit positioned on a diameter of the disk and away from the center of the disk.

7. The apparatus of claim 1 wherein the rim flange is between 5/32 and 9/32 in thickness.

8. A tissue protection and fluid collection mask apparatus comprising: a flexible and elastic sheet material providing a peripheral rim flange thereby strengthening a peripheral edge of the disk; and a linear slit, the slit providing a means for preventing slit tear-out.

9. The apparatus of claim 8 wherein the tear-out preventing means comprises two round holes, the holes located at opposing ends of the linear slit.

10. The apparatus of claim 9 wherein the holes are no larger than ¼ inch in diameter.

11. The apparatus of claim 8 wherein the elastic sheet material is one of a natural latex and silicone rubber having a maximum thickness of 3/32″.

12. The apparatus of claim 8 wherein the sheet material is between 12 and 20 inches across in at least one direction.

13. The apparatus of claim 8 wherein the slit is between 2.5 and 3.5 inches in length, and between 1/32 and 3/32 in width; the slit positioned asymmetrically on the sheet material.

14. The apparatus of claim 8 wherein the rim flange is between 5/32 and 9/32 in thickness.

15. A method of protecting tissue and collecting fluids from a surgical site comprising the steps of: preparing a disk of a flexible and elastic sheet material; providing the disk with a peripheral rim flange; opening a linear slit in the disk, the slit positioned away from a center of the disk; draping the disk over a surgical site with the slit opened around the surgical site and the bulk of the disk positioned in a down-hill direction from the surgical site; holding edges of the slit sealed against a surgical surround of the site while applying a fluid to the surgical site; and collecting overflow of the liquid from the surgical site onto the disk for disposal.
